(19) United States
(12) Patent Application Publication
(10) Pub. No.: US 2024/0175853 A1
(43) Pub. Date: May 30, 2024

(54) METHOD FOR CHARACTERIZING AN ANALYTE PRESENT IN A GAS SAMPLE CONTAINING AT LEAST ONE PARASITIC CHEMICAL SPECIES

(71) Applicant: ARYBALLE, GRENOBLE (FR)

(72) Inventor: Pierre Maho, Grenoble (FR)

(21) Appl. No.: 18/549,403

(22) PCT Filed: Mar. 4, 2022
(86) PCT No.: PCT/EP2022/055555

§ 371 Date: Sep. 7, 2023

(30) Foreign Application Priority Data
Mar. 8, 2021 (FR) FR2102231

(51) Int. Cl. G01N 33/00; G01N 21/552

(52) U.S. Cl. CPC G01N 33/0059; G01N 21/553; G01N 33/0021

(57) ABSTRACT

A method for characterizing an analyte A present in a gas sample using an electronic nose including M sensitive site, a parasitic chemical species P being present in the gas sample, the method include: a phase 100 of acquiring N first signatures, where N>1, of the gas samples containing the analyte A and the parasitic species P, the gas samples exhibiting deviations Δc_{P(n)} which differ from one gas sample to the next; a phase 200 of solving an optimization problem so as to obtain N corrected signatures, characterising the analyte A present in the N gas samples, from the N first signatures, by optimizing to objective functions.

### TECHNICAL FIELD

The field of the invention relates to that of characterising an analyte present in a gas sample by means of an electronic nose. In addition to the analyte to be characterised, the gas sample includes here at least one chemical species capable of also interacting with the functionalised measuring surface of the electronic nose.

### PRIOR ART

The ability to characterise analytes contained in gas samples, for example odorous molecules or volatile organic compounds, is an increasingly important issue, particularly in the fields of health, the agrifood industry, the perfume industry scents), olfactory comfort in confined public or private spaces (motor vehicles, hotels, shared spaces . . . ), etc . . . . The characterisation of such analytes present in a gas sample can be carried out by a characterisation system referred to as an “electronic nose”.

Various approaches to characterisation exist, which differ from one another in particular in whether it is necessary or not to “label” the analytes or receptors beforehand with a revelation agent. Unlike fluorescence measurements for example which require the use of such labels, measurements using surface plasmon resonance (SPR) and those us a Mach-Zehnder interferometer (MZI) are label-free techniques.

In an electronic nose using SPR or MZI technology, the analyte present in a gas sample interacts by adsorption/desorption with receptors located at several distinct sensitive sites of a functionalised measuring surface. It consists of detecting in real time a measurement signal associated with each of the sensitive sites, which is representative of adsorption/desorption interactions between the analyte and the receptors in response to a primary signal. The measurement signals can be optical signals representative of a temporal variation in the local refractive index due to interactions of the analyte with the receptors. In SPR technology for example the intensity of optical signals coming from different sensitive sites is measured in real time, these optical signals being a reflected part of a primary optical signal emitted by a light source. The intensity of each optical signal detected by an optical sensor is directly correlated with the absorption/desorption interactions of the analyte with the receptors. Application WO2018/158458 describes an example of such an electronic nose us SPR technology.

Insofar as the chemical and/or physical affinity of interaction of the analyte with the receptors is not known a priori, the characterisation of the analyte then comes down to determining a value or a variation of a parameter representative of the adsorption/desorption interactions of the analyte with the receptors, here representative of the temporal variation in the local refractive index for each of the sensitive sites. In this way an interaction pattern or a signature which characterises the analyte is obtained. Indeed, the adsorption/desorption interactions of the analyte or sensitive sites (functionalised surfaces) with different adsorption characteristics make it possible to account for molecules present in the gas which are attached to the surface of different sensitive sites.

FIGS. 1A and 1B illustrate an example of an SPR-type electronic nose as described in document WO2018/158458. This type of electronic nose 1 generally comprises a fluid supply device 2, a device for characterisation 3 by SPR imaging, and a processing unit (not shown).

The characterisation device 3 includes a measuring chamber 4 intended to receive the gas sample, in which a measuring surface 5 is located on which there is a matrix of sensitive sites. The measuring surface 5 is formed by a metal layer to which various receptors adapted to interact with the analyte are fixed, the receptors being arranged so as to form different sensitive sites that are distinct from one another. These receptors are then located at the interface between the metal layer and a dielectric medium, here a gaseous medium.

This characterisation device 3 further comprises a light source 7 of a primary optical signal and an image sensor 8. The light source 7 is adapted to emit the primary optical signal in the direction of the measuring surface 5, at a working angle OR allowing surface plasmons to be generated there. The reflected part of the primary optical signal, forming an optical measurement signal, is then detected by the image sensor 8. The intensity of the optical measurement signal depends locally on the refractive index of the measuring surface 5, which in turn depends on the surface plasmons generated and the amount of material located at each sensitive site, this amount of material varying over time as the analyte interacts with the receptors. The measuring surface 5 comprises a plurality of sensitive sites 6ₘ, here distinct M sensitive sites, functionalised by the presence of receptors with which the analyte to be characterised can interact by adsorption/desorption.

The processing unit of the electronic nose is suitable for analysing the “sensorgrams”, i.e. the signals corresponding to the temporal evolution of the parameter representative of the adsorption/desorption interactions of the analyte with the receptors of each of the different sensitive sites 6ₘ, with the aim of extracting therefrom information on the kinetics of interaction (adsorption and desorption) of the analyte with the receptors. These sensorgrams can be referred to as useful signals Suₘ(t) corresponding to the temporal evolution of the variation Δ%Rₘ(t) of the reflectivity associated with each of the sensitive sites 6ₘ. The reflectivity %R is here the ratio between the intensity of the optical measurement signal detected by the image sensor 8 and the intensity of the primary optical signal emitted by the light source 7. The variation in reflectivity Δ%R is obtained by subtracting from the temporal variation in reflectivity %R(t) a baseline value associated with the gas alone present inside the measuring chamber 4, independently of the analyte.

Lastly, the fluid feed device 2 is suitable for introducing the analyte into the measuring chamber 4 in conditions that allow the analysis of sensorgrams and therefore the characterisation of the analyte. In this respect, the article by Brenet et al. entitled Highly-Selective Optoelectronic Nose based on Surface Plasmon Resonance Imaging for Sensing Gas Phase Volatile Organic Compounds, Anal. Chem. 2018, 90, 16, 9879-9887, describes a method for characterising a gas sample with an electronic nose using SPR imaging. This characterisation method consists of supplying the measuring chamber with a gas sample such that the kinetics of interaction between the analyte and the receptors reach a steady-state equilibrium.

For this, FIG. 1C illustrates an example of sensorgrams Suₘ(t) obtained by the electronic

nose of FIG. 1A. The sensorgrams Suₘ(t) correspond here to the temporal evolution of the variation in reflectivity Δ%Rₘ(t) associated with each of the sensitive sites 6ₘ. Thus, the characterisation method includes a plurality of successive fluid injection steps, namely:
a first reference phase Ph1, in which a carrier gas alone, without the analyte, is brought into contact with the measuring surface. This carrier gas is generally identical to that of the gas sample;
a second feed phase Ph2, in which the gas sample, formed by the carrier gas and the analyte to be characterised, is brought into contact with the measuring surface; and
a third purge phase Ph3, in which the reference gas alone is again injected into the measuring chamber, so as to dissociate the analyte from the receptors and to remove it from the measuring chamber.

The initial phase Ph1 makes it possible to acquire the reference value (baseline) mentioned above, which is then intended to be subtracted from the measurement signals Sₘ(t) to obtain useful signals Suₘ(t) (in other words the temporal evolution of the variation in reflectivity Δ%Rₘ(t) for each sensitive site of rank m). As indicated above, this fluid injection phase is carried out such that the sensorgrams show the presence of a transitional assimilation regime followed by a steady-state equilibrium. When this steady-state equilibrium is achieved, the (stationary) equilibrium values Su_{m,f} of the useful signals Suₘ(t) are extracted by the processing unit and define the signature of the analyte.

However, it appears that the presence of other chemical species in the carrier gas, such as water molecules (relative humidity), can have an impact on the intensity of the optical measurement signal, as shown in the article by Shao et al. entitled Mechanism and Characteristics of Humidity Sensing with Polyvinyl Alcohol-Coated Fiber Surface Plasmon Resonance Sensor, Sensors 2018, 18, 2029. In this article, the authors use an SPR sensor as a humidity sensor. However, in the context of a method for characterising analytes with an electronic nose, the variation in relative humidity in the measuring chamber forms a measurement bias that degrades the quality of the characterisation. In addition, when the relative humidity varies over long periods of time and therefore varies from one characterisation to the other for the same analyte and the same operating conditions, this causes a time drift which make the signatures of the same analyte different from one another.

Furthermore, document WO 2020/141281 A1 describes a method for characterising target compounds by means of an electronic nose type analysis system.

### DESCRIPTION OF THE INVENTION

The objective of the invention is to overcome, at least partly, the disadvantages of the prior art, and more particularly to propose a method for characterising an analyte which makes it possible to limit or even eliminate the measurement noise associated with a variation in concentration of one or more parasitic chemical species present between the initial phase Ph1 and the characterisation phase Ph2 of fluid injection. The characterisation method thus makes it possible to improve the quality of characterisation of the analyte.

For this, the object of the invention is a method for characterising an analyte A present in a gas sample located in contact with a measuring surface of an electronic nose, the measuring surface including M sensitive sites distinct from one another, of rank m ranging from 1 to M, having receptors adapted to interact by adsorption/desorption with the analyte A and with at least a so-called parasitic chemical species P present in the gas sample, the method including the following steps:
fluid injection, for bringing into contact with the measuring surface: during a first phase Ph1, a carrier gas which may contain the parasitic species P in a concentration c_{P(n)i}; then, during a second phase Ph2, the gas sample containing the analyte A in concentration c_{A(n)} and the parasitic species P in a concentration c_{P(n)f}, the value c_{P(n)f}having a non-zero deviation Δc_{P(n)} from the value c_{P(n)i};
determining a measurement signal S_{(n,m)}(t), during the fluid injection step, which is representative of interactions of the analyte A and the parasitic species P with the receptors, for each of the M sensitive sites;
determining first signatures Su_{(n,m)f} from the measurement signal S_{(n,m)}(tϵPh2) associated with the gas sample, which has been corrected by a reference value S_{(n,m)i} from the measurement signal S_{(n,m)}(tϵPh1) associated with the carrier gas;
determining a difference Δc_{P(n)} in concentration of the parasitic species P between the first phase Ph1 and the second phase Ph2;
reiterating the preceding steps, incrementing the rank n until N first signatures representative of the interactions of the analyte A and the parasitic species P with the receptors are obtained, with N>1, the gas samples being such that the differences Δc_{P(n)} are different in pairs;
forming a matrix of first signatures Su_{A,P}, of dimensions N×M, formed from N first signatures Su_{(n,m)f} determined for the sensitive sites M; and a relative concentration vector Δc_{P}, of dimension N×1, from the N differences Δc_{P(n)} determined;
determining an estimated solution {\hat{k}_{P|A}; ĉ_{A}; \hat{k}_{A}}; the product of which ĉ_{A}\hat{k}_{A}^{T} forms a matrix of so-called corrected signatures Suc_{A} characterising the analyte A present in the gas samples N, the estimated solution: minimising the cost function f=Su_{A,P}-Δc_{P}k_{P|A}^{T}-c_{A}k_{A}^{T}, and maximising the cost functiong=Su_{A,P}-Δc_{P}k_{P|A}^{T}; where c_{A}, k_{A} and k_{P|A} are variables defined in the following manner:
c_{A} is a concentration vector of analyte A, of dimension N, formed from N concentration values c_{A(n)} of the gas samples,
k_{A} is an affinity vector of analyte A, of dimension M, formed from the M values of an interaction affinity of the analyte A with the receptors of the sensitive sites,
k_{P|A} is an affinity vector of the parasitic chemical species P, of dimension M, formed from the M values of an interaction affinity of the parasitic chemical species P with the receptors of the sensitive sites in the presence of the analyte A.

Some preferred but non-limiting aspects of this characterisation method are as follows.

The step of determining the estimated solution may include the minimisation of the objective function f and the maximisation of the objective function g at the same time, the values of the relative concentration vector Δc_{P} all being positive.

The step of determining the estimated solution can be performed by an iterative algorithm, of iteration indicator i.

The step of determining the estimated solution may include a substep of determining the value \hat{k}_{P|A}⁽ⁱ⁺¹⁾ of the variable k_{P|A}, given the value ĉ_{A}⁽ⁱ⁾ of the variable c_{A} and the value \hat{k}_{A}⁽ⁱ⁾ of the variable k_{A}, by a fixed point method.

The step of determining the estimated solution can include a substep of determining the value ĉ_{A}⁽ⁱ⁺¹⁾ of the variable c_{A} and the value \hat{k}_{A}⁽ⁱ⁺¹⁾ of the variable \hat{k}_{A}, given the value \hat{k}_{P|A}⁽ⁱ⁺¹⁾ of the variable k_{P|A} having been determined by a singular value decomposition of a matrix R=Su_{A,P}-Δc_{P}k_{P|A}^{T(i+1).}

The step of determining the estimated solution can include a substep of minimising the objective function f to obtain a plurality of Q local solutions minimising the objective function f, with Q>1, followed by a substep of maximising the objective function g.

The minimisation substep can provide local solutions Q, each formed by estimations \hat{k}_{P|A}^{T(q)}, ĉ_{A}^{(q)}, \hat{k}_{A}^{T(q)}, of rank q ranging from 1 to Q, of variables k_{P|A}, c_{A}, and k_{A}. Q is here greater than 1: Q>1.

The maximisation substep can include determining so-called corrected signature Q matrices Suc_{A}^{(q)} (such that: ∀qϵ[1, Q], Suc_{A}^{(q)}=Su_{A,P}-Δc_{P}\hat{k}_{P|A}^{T(q)}.

The method can include, following determining the corrected signature Q matrices Suc_{A}^{(q)}, a normalisation of each of the corrected signature matrices Suc_{A}^{(q)} to obtain corrected and normalised signature Q matrices Sucn_{A}^{(q)}.

The method can include, following the determination of the corrected and normalised signature Q matrices Sucn_{A}^{(q)}, determining a variance score, for each of the Q matrices Sucn_{A}^{(q)}, the variance score being defined as the trace of the covariance matrix for each of the Q matrices Sucn_{A}^{(q)}, the matrix Sucn_{A}^{(qf)} having a minimum score characterising the analyte A present in the gas samples N.

The method can comprise, following the determination of the corrected signature Q matrices Suc_{A}^{(q)}, determining a norm of each of the Q matrices Suc_{A}^{(q)}, followed by an identification of the matrix Suc_{A}^{(qf)} having the maximum norm.

The matrix Suc_{A}^{(qf)} can be normalised, thus providing a matrix Sucn_{A}^{(qf)} characterising the analyte A present in the gas samples N.

The electronic nose can include a device for measuring interactions by adsorption/desorption of the optical surface plasmon resonance type or of the Mach-Zehnder interferometry type.

The electronic nose can include a device for measuring interactions by resistive, piezoelectric, mechanical or acoustic-type adsorption/desorption.

The electronic nose can include a fluid supply device adapted to perform the fluid injection step, a measurement device adapted to perform the step of determining the measurement signal, a sensor for measuring and determining the relative concentration of the parasitic chemical species, and a processing unit adapted to implement the step of determining the estimated solution.

### BRIEF DESCRIPTION OF THE DRAWINGS

Other aspects, aims, advantages and features of the invention will become more apparent

upon reading the following detailed description of preferred embodiments, given by way of non-limiting example, and made with reference to the appended drawings wherein:

FIG. 1A, already described, is a schematic and partial view, in cross-section, of an SPR imaging electronic nose according to an example of the prior art;

FIG. 1B, already described, is a plan view, schematic and partial, of a functionalised measuring surface of the electronic nose of FIG. 1A including M distinct sensitive sites;

FIG. 1C, already described, is an example of sensorgrams Suₘ(t) obtained by the electronic nose of FIG. 1A, these sensorgrams corresponding here to the temporal evolution of the variation of the reflectivity Δ%Rₘ(t) associated with the sensitive sites;

FIG. 2 is a schematic and partial view of an electronic nose according to one embodiment;

FIG. 3A is an example of three signatures obtained by a characterisation method according to the prior art, showing the degradation of the characterisation of the analyte due to a variation in concentration of a parasitic chemical species (here water in the vapour phase) between the initial phase Ph1 and the characterisation phase Ph2;

FIG. 3B illustrated the temporal evolution of the concentration cp of a parasitic chemical species, of the measurement signal Sₘ(t) and of the useful signal Suₘ(t), in the event where there is no variation in the concentration cp between the initial phase Ph1 and the characterisation phase Ph2 (left-hand part), and in the event that there is a non-zero variation in the concentration cp between the initial phase Ph1 and the characterisation phase Ph2 (right-hand part);

FIG. 4 is a flowchart of a characterisation method according to a first embodiment;

FIG. 5 is a flowchart of a characterisation method according to a variant of the first embodiment;

FIG. 6 is a flowchart of a characterisation method according to a second embodiment;

FIG. 7A illustrates examples of uncorrected signatures of analyte present in gas samples for which there has been a variation in concentration cp of the parasitic chemical species;

FIG. 7B illustrates the analyte signatures of FIG. 7A corrected by the characterisation method according to the second embodiment.

### DETAILED DESCRIPTION OF PARTICULAR EMBODIMENTS

In the figures and in the remainder of the description, the same reference signs represent identical or similar elements. In addition, the various elements are not shown to scale in such a way as to give preference to the clarity of the figures. Moreover, the various embodiments and variants are not exclusive of one another and may be combined together. Unless otherwise specified, the terms “substantially”, “approximately”, “in the order of” mean to the nearest 10%, preferably to the nearest 5%. Moreover, the terms “between . . . and . . . ” and equivalent mean that the ranges are included, unless otherwise specified.

The invention relates to the characterisation of analyte A present in a gas sample to be analysed. The characterisation is performed by means of an analysis system referred to as an ‘electronic nose’, which comprises: a measuring device; a fluid feed device; a concentration sensor for the parasitic chemical species P; and a processing unit.

As detailed below, the measuring device includes a functionalised measuring surface

defining a plurality of sensitive sites, each sensitive site including receptors capable of interacting with the analyte by adsorption/desorption, and here with at least one chemical species P, different from the analyte, and therefore qualified as a parasite insofar as it induces a measurement noise on the signature of the analyte A.

By way of illustration, the electronic nose uses optical measurement technology using

silicon interferometric technology (MZI) or surface plasmon resonance (SFR). The measuring device therefore comprises an optical source and at least one optical detector which can be an image sensor or a matrix of photodetectors. The intensity of the measurement signal detected depends on the value of the local refractive index of the sensitive site in question, which is representative of interactions between the analyte A to be characterised (and the parasitic chemical species P) and the receptors.

Alternatively, other measuring technologies may be implemented, such as electromagnetic resonators of the MEMS or NEMS type (for example described in document EP3184485). More broadly, the measuring device may be of the resistive, piezoelectric, mechanical, acoustic or optical type. In the case of techniques for measuring the resonance frequency of a NEMS or MEMS microresonator, the measurement signal can be an electrical signal representative of the vibration of a microbeam or equivalent.

In general, the term “characterisation” means obtaining information representative of the interactions of the analyte A contained in the gas sample with the receptors of sensitive sites of the electronic nose. The interactions in question here are adsorption and/or desorption events of the analyte A with receptors. This information thus forms an interaction pattern, in other words a “signature” of the analyte, this pattern being represented for example in the form of a histogram or radar diagram. In other words, in the event that the electronic nose comprises M distinct sensitive sites, the signature of the analyte A is a vector of dimension M formed by the scalar representative information, which is derived from the useful signal Suₘ(t) associated with the sensitive site in question.

The analyte A is at least one chemical species intended to be characterised by the electronic nose, and is present in a gas sample. They may, by way of illustration, be bacteria, viruses, proteins, lipids, volatile organic molecules, inorganic compounds, etc. Furthermore, receptors (ligands) are elements fixed to sensitive sites and which have the ability to interact with the analyte A, although the chemical and/or physical affinities denoted ka, between the analyte A and the receptors are not initially known. The receptors of different sensitive sites have different physico-chemical properties, which affect their ability to interact with the analyte A, and thus define the different sensitive sites. For example they may include amino acids, peptides, nucleotides, polypeptides, proteins, organic polymers etc. The analyte A can be a single chemical species, or a set of different chemical species whose relative proportion remains constants (for example within 5% or 10%) from one gas sample to the other.

Within the scope of the invention, the gas sample which contains the analyte A also contains at least one so-called parasitic chemical species P as it is distinct from the analyte A to be characterised and can interact with the receptors. Its interaction by adsorption/desorption with the receptors affects the measurement signal which can then include a useful part associated with the analyte A and a non-useful part associated with the chemical species P in question and forming a measurement noise. The latter can be water molecules when the relative humidity of the gas sample is not zero, an alcohol (ethanol, butanol . . . ), hydrogen sulphide, among others. However, it appears that the variation in concentration of the parasitic species P between the initial phase Ph1 and the characterisation phase Ph2 forms a measurement noise which can degrade the quality of the characterisation of the analyte A. The characterisation method according to the invention makes it possible to eliminate this measurement noise.

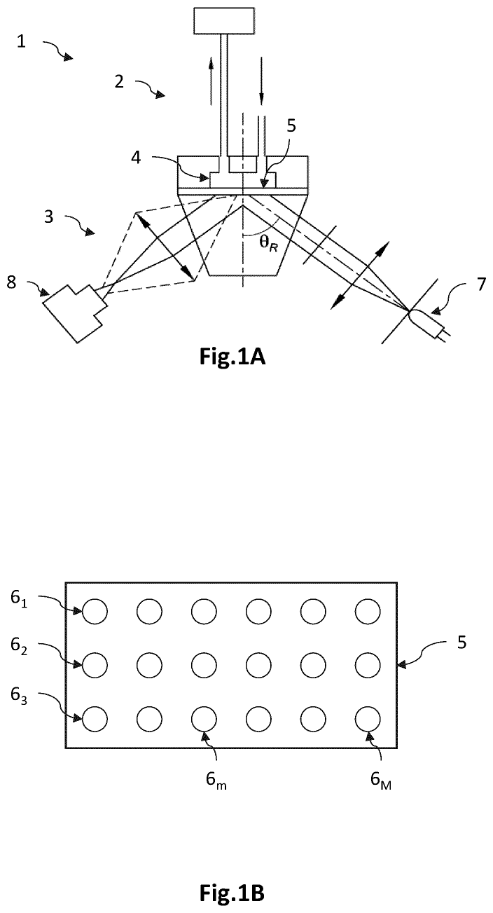

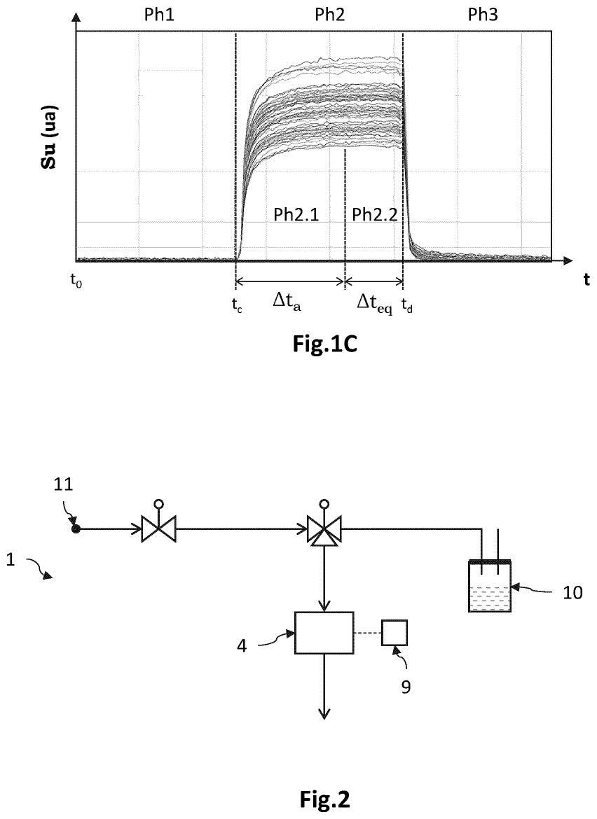

FIG. 2 is a schematic and partial view of an electronic nose 1 using SPR imaging according to one embodiment. The electronic nose 1 according to the invention can be similar to the one described with reference to FIG. 1A and FIG. 1B, and differs essentially in that it includes a sensor 9 for concentration of the parasitic chemical species P, located for example in the measuring chamber. If there is a variation in concentration of several parasitic species P₁, P₂ . . . between the fluid injection phases Ph1 and Ph2, the electronic nose can comprise a single sensor 9 or a plurality of sensors 9, adapted to measure the concentration of each parasitic species P. In the rest of the description, for the sake of clarity, it is assumed that there is only one parasitic chemical species P.

In this example, the electronic nose 1 is based on SPR technology and has in this example the features of the Kretschmann configuration, known by the person skilled in the art, without the invention being limited to this configuration. However, as indicated above, other measurement techniques can be used, such as measurements of the resonance frequency of a MEMS or functionalised NEMS type microresonator. As mentioned above, the electronic nose 1 can also be based on an optical measurement by Mach-Zehnder interferometry, for example in silicon photonics technology, as described in patent application FR2011842 filed on 18.11.2020.

The electronic nose 1 includes M sensitive sites 6ₘ, with m ranging from 1 to M, distinct from one another and located in a measuring chamber 4 intended to receive the gas sample to be analysed, these sensitive sites 6ₘ each being formed by receptors capable of interacting with the analyte A to be characterised (cf. FIG. 1B) and here with the parasitic species P. The sensitive sites 6ₘ are distinct from one another in the sense that they comprise different receptors in terms of chemical and/or physical affinity for the analyte A to be characterised, and are therefore intended to provide different interaction information from one sensitive site 6ₘ to the other. The sensitive sites 6ₘ are distinct zones of a measuring surface 5, and can be adjacent or spaced apart from one another. The electronic nose 1 can also include a plurality of identical sensitive sites, with the aim for example of detecting any measurement drift and/or enabling the identification of a defective sensitive site.

The electronic nose includes a measuring device 3, here of the SPR imaging type, making it possible to quantify the interactions of the chemical species with the receptors, for each sensitive site 6ₘ, here by measuring in real time the intensity of a measurement optical signal coming from the sensitive site 6ₘ in question, this optical signal being here a reflected part of a primary optical signal emitted by a light source 7. The intensity of the optical measurement signal detected by the optical sensor 8 is directly correlated particularly with the adsorption/desorption interactions of the chemical species with the receptors. In the case of techniques for measuring the resonance frequency of a NEMS or MEMS microresonator, the measurement signal can be an electrical signal representative of the vibration of a microbeam or equivalent.

In the context of an SPR imaging measurement, the measuring device 3 is capable of acquiring in real time the optical measurement signal Sₘ(t) coming from all of the sensitive sites 6ₘ. Thus, the optical measurement signals Sₘ(t) coming from the sensitive sites 6ₘ in response to the primary optical signal are detected together and in real time, in the form of an image acquired by the same optical sensor 8.

Thus, the optical measuring device 3 includes a light source 7 capable of transmitting a primary optical signal in the direction of sensitive sites 6ₘ, and generating surface plasmons at the level of the measurement support 5. The light source 7 can be formed by a light-emitting diode, the emission spectrum of which has an emission peak centred on a central wavelength λ_{c}. Various optical elements (lenses, polarisers . . . ) can be arranged between the light source 7 and the measurement support 5.

The optical measuring device 3 also includes an optical sensor 8, and here an image sensor, i.e. a matrix optical sensor capable of collecting or detecting an image of the optical signal coming from the sensitive sites in response to the primary optical signal. The image sensor 8 is a matrix photodetector, for example a CMOS or CCD sensor. It therefore includes a matrix of pixels whose spatial resolution is such that preferably several pixels acquire the optical measurement signal coming from the same sensitive site 6ₘ.

The processing unit (not shown) makes it possible to carry out the processing operations

described in the following as part of the characterisation method. This can include at least one microprocessor and at least one memory. It is connected to the optical measuring device 3, and more specifically to the image sensor 8. It includes a programmable processor capable of executing instructions recorded on an information recording medium. It further includes at least one memory containing the instructions necessary for implementing the characterisation method. The memory is also adapted to store information calculated at each instant of the measurement.

As described below, the processing unit is in particular configured to store and process a plurality of so-called elementary images acquired at a given sampling frequency fₑ, over a measurement period At, in order to determine a measurement signal Sₘ(tᵢ), at the current instant tᵢ, associated with the sensitive site 6ₘ. Preferably, the measurement signal Sₘ(tᵢ) corresponds, at a measurement instant tᵢ, to the average intensity of the optical signal reflected and detected by the image sensor 8 on the pixels associated with the sensitive site 6ₘ. The average optical intensity detected on the pixels can be performed for one or more images of the sensitive site 6ₘ, as described in detail below.

The fluid feed device 2 is suitable for feeding the measuring chamber 4 with a carrier gas alone (i.e. without the analyte A) during the initial phase Ph1, and with a gas sample formed by the carrier gas and analytes during the characterisation phase Ph2. The gas sample differs from the carrier gas essentially in that it includes the analyte A to be characterised: the parasitic species P is present in the gas sample, and may, or may not, be also present in the carrier gas. One or more additional gases may be present, but they are odourless in the sense that they induce substantially no response from the electronic nose 1. An example of additional gas present in the second gas sample can be the diluent in vapour phase. The analyte A can thus be stored in a liquid diluent contained in a reservoir 10. The vapour phase of the diluent and the analyte A are added to the carrier gas (for example humid air) to form the gas sample. In any case, the parasitic species P has a concentration C_{P,i} in the carrier gas which can be non-zero or even zero, and a concentration c_{P,f} which is non-zero and different from c_{P,i} in the gas sample. There is therefore a non-zero difference Δc_{P}=c_{P,f}−c_{P,i}.

In the example of FIG. 2, the fluid feed device 2 can include a carrier gas inlet 11 and a reservoir 10 of analyte A. Here, the reservoir 10 contains a diluent in which the analyte A is located. It includes a plurality of fluid lines which connect the carrier gas inlet 11 and the reservoir 10 on the one hand to the inlet of the measuring chamber 4 on the other hand and includes valves and possibly mass flow regulators. It thus makes it possible to supply the measuring chamber 4 with carrier gas (e.g. humid air with a water concentration c_{P,i}) during the initial phase Ph1 and the purge phase Ph3, and with the gas sample (e.g. humid air with a concentration of water c_{P,f}, analyte A, and diluent in vapour phase) during the characterisation phase Ph2. It can be suitable for ensuring that the concentration c_{A} of the analyte A in the measuring chamber remains constant over time. Furthermore, the electronic nose further comprises a sensor 9 for the concentration c_{P} of the parasitic species P, for example here a relative humidity sensor. The concentration sensor 9 c_{P} can be arranged in the measuring chamber or upstream or downstream thereof. It is connected to the processing unit, which is capable of calculating the variation in concentration Δc_{P}=c_{P,f}−c_{P,i} (or relative concentration) between the initial phase Ph1 and the characterisation phase Ph2.

However, it appears that the variation in concentration cp of the parasitic species P present

in the measuring chamber during the fluid injection step (phases Ph1 and Ph2) causes a measurement noise which degrades the quality of the characterisation. This measurement noise is a noise linked to a non-zero difference in concentration cp within the measuring chamber between the initial phase Ph1 and the characterisation phase Ph2. It consists of a measurement noise in that it arises from a temporal variation in a parameter which characterises the environment inside the measuring chamber and should normally remain stationary over time.

This problem of measurement noise related to ≢c_{P} is particularly important when the characterisation method is carried out on the basis of useful signals Suₘ(t), i.e. when it includes a step of subtracting the reference value S_{m,i} (baseline) from the corresponding measurement signal Sₘ(t). Indeed, the aim of this step is to remove from the characterisation of the analyte A the effect associated with their environment and in particular the effect of the carrier gas. However, it appears that this reference value S_{m,i} is representative of the carrier gas during the initial phase Ph1, but is no longer necessarily representative of the carrier gas during the characterisation phase Ph2, since the physical properties of this carrier gas in the measuring chamber may have changed (variation in the concentration cp of the parasitic species P).

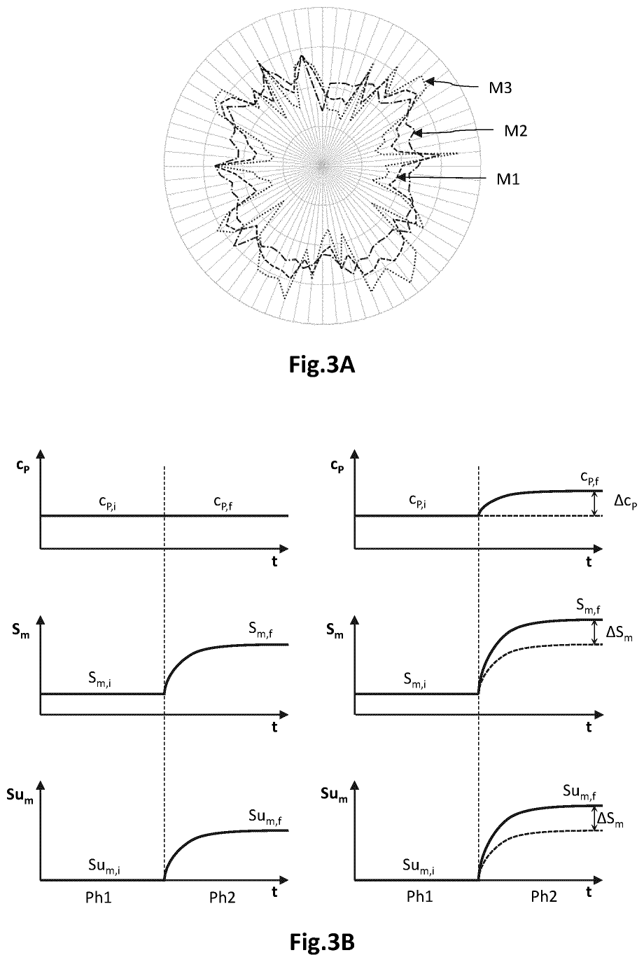

FIG. 3A illustrates three interaction patterns or signatures, reflecting the characterisation of different gas samples, this characterisation being carried out by a characterisation method according to an example of the prior art. These signatures M1, M2 and M3 are here representations in the form of a radar diagram of equilibrium values (stationary) Su_{m,f} determined by the sensorgrams Suₘ(t) in the steady-state equilibrium Ph2.2 (cf. FIG. 1C). They highlight the effect of the variation in concentration cp between the fluid injection phases Ph1 and Ph2, here a relative concentration Δc_{P}, on the characterisation of the analyte A. To obtain these signatures M1, M2, M3, the carrier gas is identical for the three tests and corresponds to humid air having an initial relative humidity c_{P,i} of about 12%.

A first signature M1 corresponds to a gas sample formed of humid air with a relative humidity c_{P,f} equal to about 50% and for which the analyte A is butanol molecules. The implementation of the characterisation method thus has a relatively large variation in relative humidity in the measuring chamber, which passes here from c_{P,i} equal to approximately 12% during the initial phase Ph1, to c_{P,f} equal to approximately 50% during the characterisation phase. Also the reference value S_{m,i} is determined for the carrier gas (humid air at a c_{P,i} of 12%) and the equilibrium value is determined for the gas sample (humid air at c_{P,f} of 50% with analyte A) by subtracting this reference value S_{m,i}. This relative concentration Δc_{P} thus forms a measurement noise, the effect of which has to be limited so that the signature M1 is effectively representative only of butanol molecules.

A second signature M2 corresponds to a gas sample formed by humid air with a relative humidity c_{P,f} substantially equal to c_{P,i} (i.e. 12%), and the analyte A of which is also butanol molecules. The implementation of the characterisation method makes it possible, by subtracting the reference value S_{m,i}, associated with the carrier gas (humid air at c_{P,i}), and insofar as the variation in relative humidity Δc_{P} is zero, to eliminate the effect of the gaseous environment and thus characterise the interactions of the analyte A with the receptors alone. Also, the signature M2 is representative of the analyte A alone since there is no measurement noise associated with the variation in relative humidity Δc_{P}. It is noted that the signature M1 is not superimposed on the signature M2, reflecting the presence of the measurement noise associated with Δc_{P} in the case of M1. It is therefore important to be able to correct the signature M1 in order to move towards signature M2, which alone is representative of the analyte, even if there is a difference in relative humidity Δc_{P} in the measuring chamber between the between the initial phase Ph1 and the characterisation phase Ph2.

The third signature M3 corresponds to a gas sample formed only by humid air with a relative humidity c_{P,f} equal to approximately 50%. Here, the impact alone of the variation in relative concentration Δc_{P} on the characterisation of humid air is measured by the electronic nose, in the absence of analyte. It appears that the increase in the relative concentration Δc_{P} between the initial phase Ph1 and the characterisation phase Ph2 results in an increase in the variation of reflectivity Δ%Rₘ of sensitive sites 6ₘ. It can be seen that the signature M1 (humid air with non-zero Δc_{P} and presence of the analyte A) is located between the signature M2 (humid air with zero Δc_{P} and presence of the analyte A) and the signature M3 (humid air with non-zero Δc_{P} and absence of the analyte A), clearly showing the effect of the measurement noise associated with the non-zero relative concentration Δc_{P} on the signature of the analyte A. It is therefore important to be able to limit or even eliminate this measurement noise in order to improve the quality of the characterisation of the analyte A.

FIG. 3B illustrates examples of the temporal evolution of the concentration c_{P} of the parasitic species P present in the measuring chamber during the initial phase Ph1 and the characterisation phase Ph2, of the measurement signal Sₘ(t) for the sensitive site 6ₘ of rank m, and lastly of the useful signal Suₘ(t).

The left-hand side of the graphs corresponds to the situation in which the concentration c_{P}(t) remains constant between the two fluid injection phases Ph1 and Ph2. In the initial phase Ph1, only the carrier gas is present in the measuring chamber: the parasitic species P has a concentration of value c_{P,i}, resulting in a measurement signal Sₘ(t) with a reference value S_{m,i}. In phase Ph2, the gas sample to be analysed is introduced into the measuring chamber. Due to the presence of the analyte A and as the concentration c_{P}(t) remains constant, the measurement signal Sₘ(t) moves towards a stationary value S_{m,f} greater than S_{m,i}. To obtain the useful signal Suₘ(t), the measurement signal Sₘ(t) is subtracted from its initial value S_{m,i}, such that the signature is the vector Su formed from the M values Su_{m,f} obtained.

The right-hand side of the graphs corresponds to the situation in which the concentration c_{P}(t) varies between the two fluid injection phases Ph1 and Ph2. Thus, during the initial phase Ph1, only the carrier gas is present in the measuring chamber: the parasitic species P has a concentration of value c_{P,i}, which results in a measurement signal Sₘ(t) with a reference value S_{m,i}. However, in the phase Ph2, the introduction of the gas sample causes the concentration c_{P}(t) of the parasitic species P to vary, in this case increasing to a value c_{P,f}, greater than Δc_{P} relative to the reference value c_{P,i}.

This variation in the concentration of the parasitic species P results in a variation in the measurement signal Sₘ(t) during this phase Ph2, which moves here to a value S_{m,f} greater than ΔSₘ than in the case where there is no variation in the concentration c_{P} (dotted curve). It then follows that the useful signal Suₘ(t) has a measurement noise of value ΔSₘ which depends on the difference Δc_{P}. It is then a matter, for characterising the analyte A, of evaluating the measurement noise ΔSₘ and subtracting it from the measurement signal Sₘ(t) with the reference value S_{m,i}.

To subtract the measurement noise ΔSₘ(Δc_{P}) from the measurement signal Sₘ(t), one approach can be to perform a calibration step prior to the characterisation step. Patent application FR1913555 filed on 29 Nov. 2019 describes a characterisation method including such a calibration step. The prior calibration involves determining a correction function expressing the change in the reference value S_{m,i} of the measurement signal Sₘ(t) associated with the parasitic species P alone (i.e. without the analyte A) as a function of its concentration c_{P}. Thus, in the characterisation step, instead of subtracting from the stationary value S_{m,f} the reference value S_{m,i} determined during the initial phase Ph1 in which the concentration c_{P} has the c_{P,i}, the value S_{m,i}, from the correction function corresponding to the effective value c_{P,f} during phase Ph2 is subtracted.

However, it appears that the affinity k_{P} of the parasitic species P with the receptors can be affected by the presence of the analyte A. For example, when the parasitic species P is water molecules (water in vapour phase) and the analyte A is hydrophilic or hydrophobic, the analyte A adsorbed on the receptors can induce a force of attraction or repulsion to the water molecules, thereby modifying the affinity k_{P} of the parasitic species P. The affinity k_{P|A} is then noted as being the affinity of the parasitic species P in the presence of the analyte A. The step of calibration may then not accurately estimate the actual measurement noise during the characterisation phase Ph2 insofar as the interactions of the parasitic species P with the receptors are influenced by the presence of the analyte A.

Also, the characterisation method according to the invention is based on the idea of estimating the measurement noise from interactions of the parasitic species P with the receptors in the presence of the analyte A, and not, as in the previous calibration approach, in the absence of the analyte A. For this, the characterisation method initially involves acquiring stationary values Su_{(n,m)f} of the useful signal Su_{(n,m)}(t) for N different gas samples, which contain the same chemical species, i.e. the same analyte A and the same parasites species P, but for which the relative concentration Δc_{P(n)} is different from one acquisition n to the next n+1. Then a matrix of first signatures Su_{A,P} (i.e. uncorrected signatures) is obtained which is representative of interactions of the analyte A and the parasitic species P with the receptors, and a relative concentration vector Δc_{P} of the chemical species P is obtained during the acquisitions N. In a second step, an optimisation problem is solved, using the matrix of first signatures Su_{A,P} and the relative concentration vector Δc_{P}, to obtain a matrix of corrected signatures Sucn_{A}, which are representative only of the interactions of the analyte A with the receptors during acquisitions N. These corrected signatures then provide a characterisation of the analyte A for each of the gas samples.

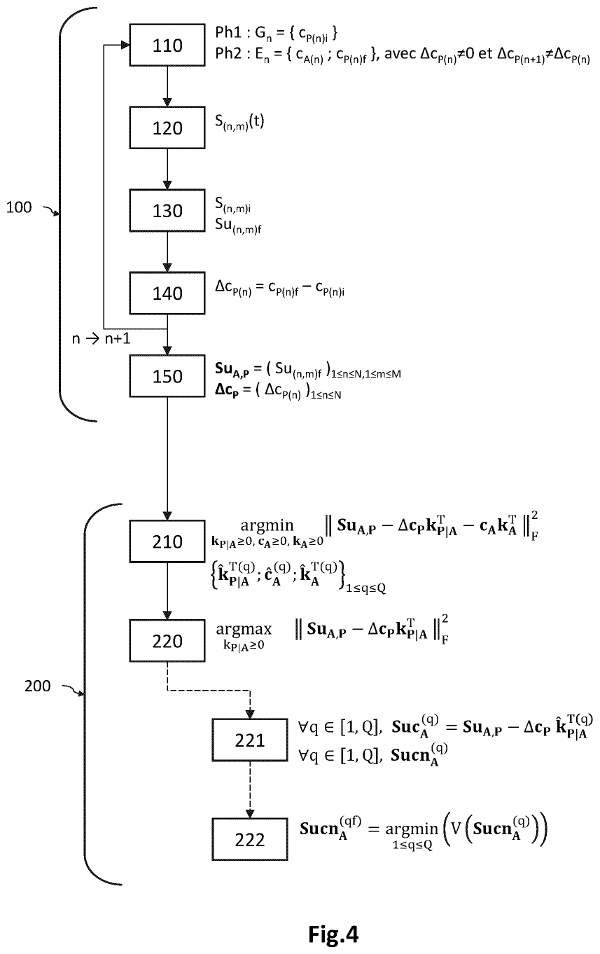

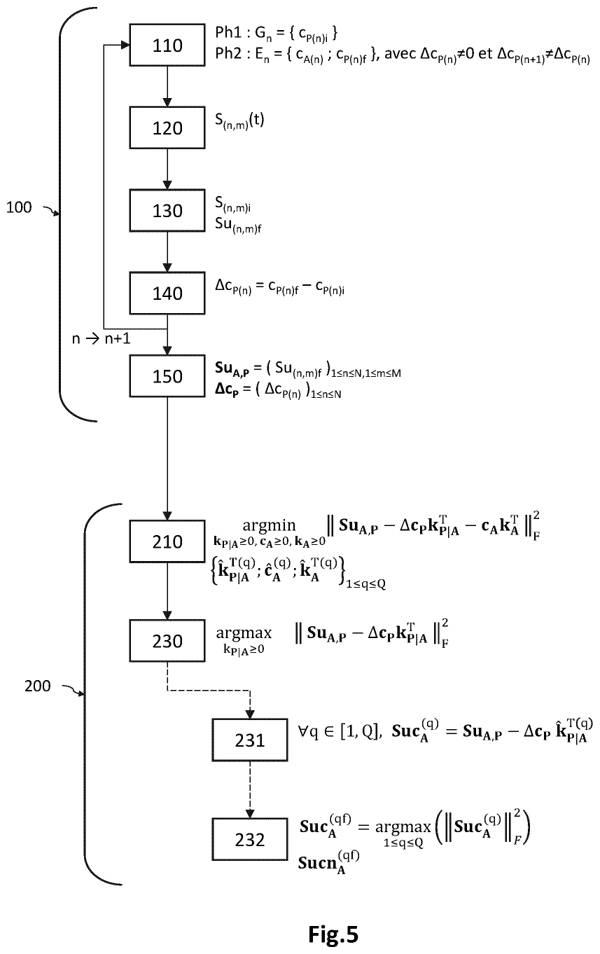

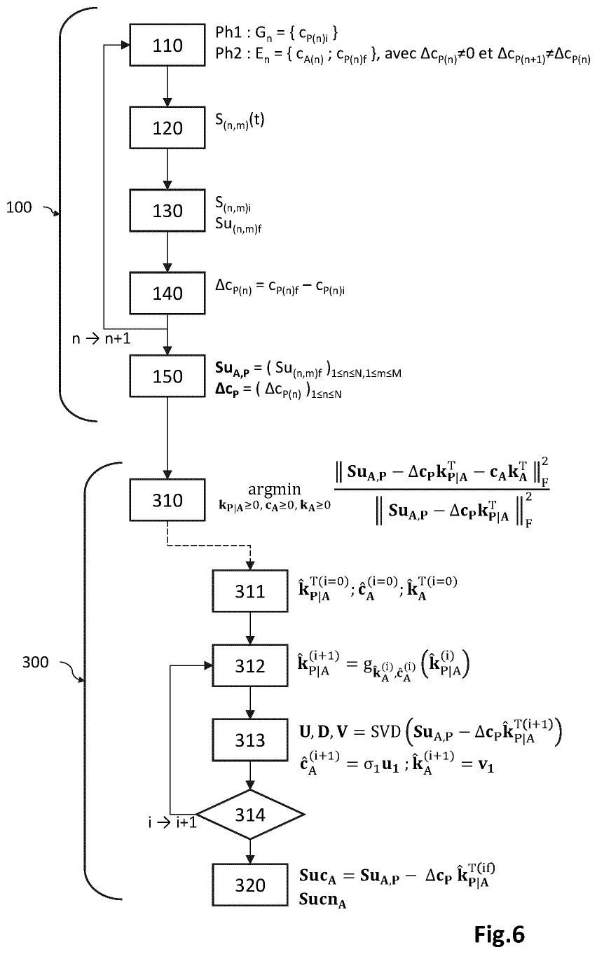

In the following examples of a characterisation method, this optimisation problem can be solved in two stages: this is the first embodiment (flowcharts in FIG. 4 and the FIG. 5). Alternatively, it can be solved in one step: this is the second embodiment (flowchart in FIG. 6). Other methods of solving this optimisation problem are possible of course.

FIG. 4 is a flowchart of a method for characterising the analyte A according to a first embodiment, which makes it possible to improve the quality of characterisation of analyte A by reducing or even eliminating the measurement noise associated with the non-zero relative concentration Δc_{P} between the injection phases Ph1 and Ph2. Analyte A is contained in gas samples, the latter also containing at least one parasitic chemical species P. In this example, the parasitic species P is water molecules, but may be one or more other parasitic species, such as ethanol and hydrogen sulphide, among others.

As indicated above, in a first phase 100, N first signatures (uncorrected signatures) of gas samples are acquired, which are therefore representative of the interactions of the analyte A and the parasitic species P with the receptors. Then, in a second phase 200, an optimisation problem is solved so as to estimate the contribution of the parasitic species P in the first signatures, in order to obtain the corrected signatures characterising the analyte A alone.

Phase 100: acquisition of first signatures N, with N>1, of gas samples containing the analyte A and the parasitic species P.

The following steps 110 to 140 are carried out N times, with N>1, each time for a different gas sample, these N gas samples containing the analyte A and the parasitic species P, and differing from one another at least by the concentration c_{P} of the parasitic species P during the injection phase Ph2. Each acquisition phase is denoted by an indicator n, a non-zero integer ranging from 1 to N. It should be noted that the greater the value of N, the better the quality of the estimate of the water signature and therefore the correction of the first signatures.

In a first step 110, a carrier gas G₍ₙ₎ is injected into the measuring chamber: this is the fluid injection phase Ph1 mentioned above. The carrier gas G₍ₙ₎ does not contain the analyte A. It may or may not contain the parasitic species P: the concentration c_{P(n)i} may therefore be non-zero or zero. It may also include other chemical species, but which do have adsorption/desorption type interactions with the receptors of sensitive sites 6ₘ: so they are not so-called parasite species.

The gas sample E₍ₙ₎ is then injected into the measuring chamber: this is the fluid injection phase Ph2 mentioned above. The gas sample E₍ₙ₎ of rank n includes the analyte A in concentration c_{A(n)} and the parasitic species P in concentration c_{P(n)f}. Here again, the gas sample may include chemical species which do not interact with the receptors. Also, only the analyte A and the parasitic species P interact with the receptors and cause a variation in the measurement signal S_{(n,m)}(t).

In step 120, in parallel with step 110, the measurement signals S_{(n,m)}(t) associated with the sensitive sites 6ₘ are determined, with m ranging from 1 to M, with M>1. These measurement signals S_{(n,m)}(t) are therefore representative, in the fluid injection phase Ph1 (carrier gas G₍ₙ₎ alone), of the interactions of the parasitic species P, and then in the fluid injection phase Ph2 (gas sample E₍ₙ₎), of the interactions of the analyte A and the parasitic species P.

In step 130, the reference value S_{(n,m)i} (baseline) associated with the carrier gas is determined in the injection phase Ph1, and subtracted from the stationary value S_{(n,m)f} of the measurement signal S_{(n,m)}(t) determined in the injection phase Ph2. Thus a signature Su_{(n,m)f}=S_{(n,m)f}−S_{(n,m)i} is obtained for each sensitive site m, associated with the gas sample E₍ₙ₎ for the acquisition of rank n.

In step 140, the value c_{P(n)i} of the concentration of the species P in the injection phase Ph1 is measured, then the value c_{P(n)f}in the injection phase Ph2, and the difference Δc_{P(n)}=c_{P(n)f}−c_{P(n)i} in concentration (or relative concentration) is determined.

The preceding steps are repeated N times, so as to obtain N first signatures Su_{(n,m)f} for N ranging from 1 to N, and m ranging from 1 to M. From one iteration to the other, therefore between n and n+1, the corresponding gas samples E₍ₙ₎ and E₍ₙ₊₁₎ differ from one another essentially by the difference Δc_{P(n)}≈Δc_{P(n+1)}: there are therefore N differences Δc_{P} with values different from one another. It should be noted that the concentration c_{A} of analyte A may vary, or may not vary, from one gas sample to the other.

In step 150, a matrix of first signatures Su_{A,P} is formed which is representative of the interactions of the analyte A and the parasitic species P with the receptors of the sensitive sites. This matrix is of dimension N×M and is formed by the values Su_{(n,m)f}. In other words, each row of rank n represents the signature associated with the gas sample E₍ₙ₎, i.e. the M useful measurement values Su_{(n,m)f} of the sensitive sites 6ₘ. A relative concentration vector Δc_{P} is also formed from the N determined values of the concentration difference Δc_{P(n)}. This vector is here a vector column of dimension N×1.

Phase 200: solving an optimisation problem in order to obtain N corrected signatures, characterising the analyte A present in the N gas samples.

In general, an estimated solution is sought {\hat{k}_{P|A}; ĉ_{A}; \hat{k}_{A}}; the product of which ĉ_{A}\hat{k}_{A}^{T} forms a matrix of corrected signatures Suc_{A} characterising the analyte A present in the N gas samples, the estimated solution minimising the cost function f=Su_{A,P}-Δc_{P}k_{P|A}^{T}-c_{A}k_{A}^{T}, and maximising the objective function g=Su_{A,P}-Δc_{P}k_{P|A}^{T}. The variables in this optimisation problem are c_{A}, k_{A} and k_{P|A}, where:
c_{A} is a concentration vector of the analyte A, of dimension N×1, formed from N concentration values c_{A(n)} of the gas samples;
k_{A} is an affinity vector of the analyte A, of dimension M, formed from the M values of an interaction affinity of the analyte A with the receptors of the sensitive sites;
k_{P|A} is an affinity vector of the parasitic chemical species P, of dimension M, formed from the M values of an interaction affinity of the parasitic chemical species P with the receptors of the sensitive sites in the presence of the analyte A.

In step 210, the following optimisation problem is solved:

$${arg min}_{{k}_{P \vert A} \geq 0 , {c}_{A} \geq 0 , {k}_{A} \geq 0} {‖ {Su}_{A , P} - Δ {c}_{P} {k}_{P \vert A}^{T} - {c}_{A} {k}_{A}^{T} ‖}_{F}^{2}$$

In other words, solutions are determined, here local solutions, that minimise the cost function f such that: f=Su_{A,P}−Δc_{P}k_{P|A}^{T}−c_{A}k_{A}^{T}. This involves minimising the squared error, here in the sense of the Frobenius norm, between the matrix of first signatures Su_{A,P}and the term Δc_{P}k_{P|A}^{T}+c_{A}k_{A}^{T}. As indicated above, the terms c_{A}, k_{A} and k_{P|A}, are the variables to be optimised, and the terms Su_{A,P} and Δc_{P} are data that has been determined in step 150. The function f (and the function g) is referred to here as “cost function”, but can also be referred to as “cost”, “objective function”, “objective”, or even “criterion”. It should be noted that minimising a cost function f is equivalent to maximising the function -f.

Various known techniques for solving optimisation problems can be implemented, for

example gradient descent on all the parameters or block gradient descent where each parameter is optimised by considering the other parameters as fixed. In the two cases, it is necessary to start from a random selection of the initial conditions due to the non-convexity of the objective function which makes it difficult to find the optimum. Then a plurality of local solutions are obtained, noted {\hat{k}_{P|A}^{T(q)};ĉ_{A}^{(q)};\hat{k}_{A}^{T(q)}}_{1≤q≤Q}. In other words, Q local solutions are obtained, each formed by estimated vectors\hat{k}_{P|A}^{T(q)}, ĉ_{A}^{(q)}, \hat{k}_{A}^{T(q)}.

In step 220, the following optimisation problem is solved:

$${arg max}_{{k}_{P \vert A} ∈ {\{ {k}_{P \vert A}^{T ( q )} \}}_{1 \leq q \leq Q}} {‖ {Su}_{A , P} - Δ {c}_{P} {k}_{P \vert A}^{T} ‖}_{F}^{2}$$

This consists here of determining the solution, from the local solutions {\hat{k}_{P|A}^{T(q)}}_{1≤q≤Q} determined previously, maximising the cost function g such that g=Su_{A,P}−Δc_{P}k_{P|A}^{T}.

For this, in step 221, a corrected signature matrix Suc_{A}^{(q)} is determined for each of the Q local solutions: ∀qϵ[1, Q], Suc_{A}^{(q)}=Su_{A,P}−Δc_{P}\hat{k}_{P|A}^{T(q)}. Thus, by subtracting the estimated contribution Δc_{P}\hat{k}_{P|A}^{T(q)} of the parasitic species P in the matrix of first signatures Su_{A,P}, only the useful contribution is retained c_{A}\hat{k}_{A}^{T(q)}, i.e. the one linked to the analyte A. It is then necessary to determine which solution out of the determined Q local solutions, maximises the useful contribution c_{A}\hat{k}_{A}^{T(q)} (and therefore the cost function g).

Also each of the Q corrected signature matrices Suc_{A}^{(q)} are normalised to obtain Q corrected and normalised signature matrices Sucn_{A}^{(q)}. Thus, it is calculated for each of the corrected signatures of rank n: Sucn_{A(n)}^{(q)}=Suc_{A(n)}^{(q)}/∥Suc_{A(n)}^{(q)}∥₂.

In step 222, it is then determined which solution qf, out of the Q local solutions, has a minimum variance score. For this, a variance score V is calculated for each of the Q matrices Sucn_{A}^{(q)}, and the one with a minimum score is identified. The variance score is here the trace the covariance matrix for each of the local solutions, i.e. the sum of the variance of each sensor m for each local solution q. More precisely, the variance score can be to within one multiplicative factor:

V(Sucn_{A}^{(q)})=Trace((Sucn_{A}^{(q)i -I}_{N}μ^{T(q)})^{T}(Sucn_{A}^{(q)}−I_{N}μ^{T(q)}))

where I_{N} is a unit vector of dimension N, and where μ^{T}^{_{(q)}} is a vector of dimension M where each value m is the average of the normalised corrected signatures (Sucn_{A(n,m)}^{(q)})_{1≤n≤N} for the sensitive site 6ₘ of rank m. The solution of rank qf, out of the Q local solutions, then corresponds to the vectors ĉ_{A}^{(qf)} and \hat{k}_{A}^{T(qf)} the product of which ĉ_{A}^{(qf)}\hat{k}_{A}^{T(qf)} is equal to the corrected signature matrix Sucn_{A}^{(qf)}.

Thus, by way of the determination method according to this first embodiment, the signatures Sucn_{A}^{(qf)} are determined which characterise effectively and uniquely the analyte A for the N gas samples, despite the presence of the parasitic species P, and without resorting to a prior calibration step. The characterisation method then provides more accurate signatures of analyte A since the measurement noise Δc_{P}k_{P|A}^{T} is estimated in the presence of the analyte A and not in the absence of the latter (which is the case in the prior calibration).

FIG. 5 is a flowchart of a characterisation method of the analyte A according to one variant of the first embodiment. This variant differs from the method of FIG. 4 essentially in the manner of optimising the cost function g.

The method includes a phase 100 of acquiring N first signatures. This phase 100 can be identical or similar to the one described with reference to FIG. 4. It may therefore include steps 110 to 150, and is therefore not given in detail again here.

It then includes a phase 200 of solving an optimisation problem consisting of minimising the cost function f and then maximising the cost function g. The step 210 of minimising the cost function f may be identical to that described previously and is not explained again here.

Step 230 consists of maximising the cost function g from the Q local solutions obtained after step 210. This consists here of determining the solution, from the local solutions determined previously, maximising the cost function g such that g=Su_{A,P}−Δc_{P}k_{P|A}^{T}.

In step 231, the corrected signature matrix Suc_{A}^{(q)} is determined for each of the Q local solutions: ∀qϵ[1, Q], Suc_{A}^{(q)}=Su_{A,P}-Δc_{P}k_{P|A}^{T(q)}. This step is identical to step 221. However, unlike step 221, the Q corrected signature matrices Suc_{A}^{(q)} are not normalised.

In step 232, it is then determined which solution (of rank denoted qf), among the Q local solutions, has a maximum norm, here in the sense of the Frobenius norm. For this, the norm ∥Suc_{A}^{(q)}∥_{F}² is calculated for each of the Q local solutions, and the one with the maximum norm is identified. As before, the solution of rank qf, out of the Q local solutions, then corresponds to the vectors ĉ_{A}^{(qf)} and \hat{k}_{A}^{T(qf)} the product of which ĉ_{A}^{(qf)}\hat{k}_{A}^{T(qf)} is equal to the corrected signature matrix Suc_{A}^{(qf)}.

Then the corrected signature matrix Suc_{A}^{(qf)} is normalised: ∀nϵ[1, N], Sucn_{A(n)}^{(qf)}=Suc_{A(n)}^{(qf)}/∥Suc_{A(n)}^{(qf)}∥₂. Thus, by way of the determination method according to this embodiment, the signatures Sucn_{A(n)}^{(qf)} are determined which characterise effectively and uniquely the analyte A for the N gas samples without having to resort to a prior calibration step. The characterisation method then provides more accurate signatures of the analyte A.

FIG. 6 is a flowchart of a characterisation method of the analyte A according to a second embodiment. This method differs from the methods of FIG. 4 and FIG. 5 essentially in the manner of optimising the two cost functions f and g.

The method includes a phase 100 of acquiring N first signatures. This phase 100 can be identical or similar to the one described with reference to FIG. 4. It may include steps 110 to 150, and is therefore not described in detail again here.

It then includes a phase 300 of solving an optimisation problem consisting at the same time of minimising the cost function f=Su_{A,P}-Δcₚk_{P|A}^{T}−c_{A}k_{A}^{T} and maximising the cost function g=Su_{A,P}−Δcₚk_{P|A}^{T}). This amounts to minimising the following function J:

$${arg min}_{{k}_{P \vert A} \geq 0 , {c}_{A} \geq 0 , {k}_{A} \geq 0} \frac{{‖ {Su}_{A , P} - Δ {c}_{P} {k}_{P \vert A}^{T} - {c}_{A} {k}_{A}^{T} ‖}_{F}^{2}}{{‖ {Su}_{A , P} - Δ {c}_{P} {k}_{P \vert A}^{T} ‖}_{F}^{2}} = {arg min}_{{k}_{P \vert A} \geq 0 , {c}_{A} \geq 0 , {k}_{A} \geq 0} J ( {k}_{P \vert A} , {c}_{A} , {k}_{A} )$$

This optimisation problem is solved here by an iterative algorithm with a convergence condition. Thus, after an initialisation step 311, steps 312 and 313 are performed iteratively, until a convergence criterion is verified. There is therefore no need to perform a large number of random initialisations, as each initialisation leads to the global solution (no local solutions). It should be noted here that for this the values of the vector Ac_{P} are all positive (increase in concentration c_{P(n)} between the fluid injection phase Ph1 and the fluid injection phase Ph2).

Thus, in step 311, for the iteration indicator i=0, the iterative algorithm is initialised by assigning an initial value to the three variablesk \hat{k}_{P|A}^{T(i=0)}, ĉ_{A}⁽ⁱ⁼⁰⁾, \hat{k}_{A}^{T(i=0)}. These initial values can be selected in any way.

In step 312, the value à i+1 (here i=1) of the variable \hat{k}_{P|A}⁽ⁱ⁺¹⁾ is then determined taking into account the values at i (here i=0) of variables ĉ_{A}⁽ⁱ⁾, \hat{k}_{A}⁽ⁱ⁾. The fixed point method is used here, insofar as it appears that writing that the Jacobian of the function J as a function of the variable k_{P|A} is equal to zero is an equation of form “x=h(x)”:

$$\frac{\partial J ( {k}_{P \vert A} , {c}_{A} , {k}_{A} )}{\partial {k}_{P \vert A}} = 0 ⇔ {k}_{P \vert A} = \frac{S {u}_{A , P}^{T} Δ {c}_{P}}{Δ {c}_{P}^{T} Δ {c}_{P}} - \frac{{k}_{A} {c}_{A}^{T} Δ {c}_{P}}{Δ {c}_{P}^{T} Δ {c}_{P} ( 1 - J ( {k}_{P \vert A} , {c}_{A} , {k}_{A} ) )} = {h}_{{k}_{A} , {c}_{A}} ( {k}_{P \vert A} )$$

Also, the equation is solved, for example iteratively: \hat{k}_{P|A}ⁱ⁺¹⁾=h_{\hat{k}}_{_{A}}_{i, c}_{_{A}}ᵢ(\hat{k}_{P|A}⁽ⁱ⁾) so as to obtain the value \hat{k}_{P|A}⁽ⁱ⁺¹⁾. The method amounts to successively applying the function h until convergence.

In step 313, the values at i+1 (here i=1) of the variables ĉ_{A}⁽ⁱ⁺¹⁾, \hat{k}_{A}⁽ⁱ⁺¹⁾ are then determined, taking into account the value\hat{k}_{P|A}⁽ⁱ⁺¹⁾ which has just been determined. To achieve this, a Singular Value Decomposition (SVD) of the residue matrix R is performed such that R=Su_{A,P}-Δc_{P}k_{P|A}^{T}. In this example, N is greater than M: N>M. Thus, the residue matrix R can be factorised in the form: R=UΣV^{T}, where U is a matrix of dimensions N×M whose first column uₘ of rank m is proportional to the concentration vector c_{A} of the analyte A, where Σ is a matrix of dimensions M×M the diagonal elements of which (σₘ)_{1≤m≤M} are the singular values of the residue matrix R, and where V is a matrix of dimensions M×M the first column vₘ of rank m of which is proportional to the affinity vector k_{A}.

Also the approximation σ₁u₁v₁^{T} of rank 1 of the singular value decomposition of the residue matrix R is equal to ĉ_{A}⁽ⁱ⁺¹⁾\hat{k}_{A}⁽ⁱ⁺¹⁾. It is therefore ĉ_{A}⁽ⁱ⁺¹⁾=σ₁u₁ and \hat{k}_{A}^{(i+1)=v}₁.

In step 314, it is determined whether or not a convergence criterion is verified. This may involve comparing the variation between i and i+1 of one and/or other of the variables with a predefined threshold value. When this variation is greater than this threshold, steps 312 and 313 are repeated by incrementing the indicator i by one unit. Conversely, when this variation is less than or equal to this threshold, the next step is performed.

In step 320, the corrected signature matrix Suc_{A} is defined as follows: Suc_{A}=Su_{A,P}−Δc_{P}\hat{k}_{P|A}^{T(if)}, where if is the value of the indicator i when the convergence criterion is verified. Thus, the matrix of corrected signatures Suc_{A} is defined as being equal to the matrix of first signatures Su_{A,P} from which the estimate Δc_{P}\hat{k}_{P|A}^{T(if)} of the term representative of the measurement noise (impact of parasite species) has been subtracted. The Suc_{A} matrix can then be normalised to obtain the Sucn_{A} matrix, as indicated above in steps 221 and 232.

Thus, by way of the determination method according to this embodiment, the signatures which effectively characterise analyte A for the N gas samples have been determined without resorting to a prior calibration step. The characterisation method therefore provides more accurate signatures Sucn_{A} of the analyte A. It should also be noted that the method according to this second embodiment has the advantage of obtaining the optimum solution in a single step, insofar as the minimisation of the objective function f and the maximisation of the objective function g are carried out together to obtain the matrix of corrected signatures Suc_{A}.

It should be noted that the differences Δc_{P(n)} determined during N acquisitions of phase 100, may have a greater or lesser amplitude of variation, this amplitude of variation being defined as the difference between the maximal deviation max(Δc_{P(n)}) and the minimal deviation min(Δc_{P(n)}) among). For a small amplitude of variation, for example of in the order of 10%, it is advantageous to use the characterisation method according to the second embodiment which then gives more accurate results.

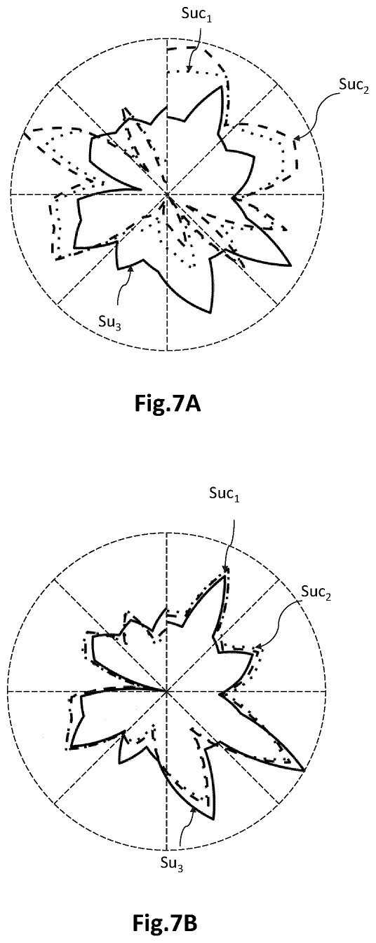

FIG. 7A illustrates three uncorrected signatures Su₁, Su₂ and Su₃ representative of the analyte A in the presence of a parasitic species P of different concentrations. In these examples, the analyte A is butanol and the parasitic species P is water in the vapour phase (humid air). The signature Su₁ (dotted line) corresponds to a deviation Δc_{P,1} of 36.5%, the signature Su₂ (dashed line) corresponds to a deviation Δc_{P,2} of 33.5%, and the signature Su₃ (solid line) corresponds to a deviation Δc_{P,3} of −1%.

FIG. 7B illustrates two corrected signatures Suc₁ and Suc₂ obtained by correcting signatures Su₁ and Su₂ by means of the second phase 300 of the characterisation method according to the second embodiment. The uncorrected signature Su₃ is reproduced here for comparison (but is not used in the optimisation). Note that a small deviation Δc_{P} has little impact on the signature Su due to the subtraction of the reference value S_{m,f}. It can be seen that the corrected signatures Suc₁ (dotted line) and Suc₂ (dashed line) are substantially merged and close to the signature Su₃ (solid line), thus illustrating the fact that the impact linked to the interactions of the parasitic species P with the receptors has been corrected.

Particular embodiments have been described above. Various variants and modifications will

become apparent to the person skilled in the art.

Thus, as indicated above, the gas samples used during the acquisition phase 100 can include a plurality of parasitic chemical species: P₁, P₂ . . . Also, during step 140, the concentration of these different parasitic species is measured during the injection phases Ph1 and Ph2, then the deviations Δc_{P1}, Δc_{P2} . . . are determined. The correction phases 200 and 300 are then similar to those described above, and are based on the optimisation of the objective functionsf=Su_{A,P}-c_{A}k_{A}^{T}-Δc_{P1}k_{P1|A}^{T}-Δc_{P2}k_{P2|A}^{T} . . . and g=Su_{A,P}-Δc_{P1}k_{P1|A}^{T}-Δc_{P2}k_{P2|A}^{T} . . .

What is claimed is:

1. A method for characterising an analyte A present in a gas sample located in contact with a measuring surface of an electronic nose, the measuring surface including M sensitive sites distinct from one another, of rank m ranging from 1 to M, having receptors configured to interact by adsorption/desorption with the analyte A and with at least one parasitic chemical species P present in the gas sample, the method including the following steps:
fluid injection, for contacting with the measuring surface:
during a first phase Ph1, a carrier gas which may contain the parasitic species P with a concentration c_{P(n)i}; then
during a second phase Ph2, the gas sample containing the analyte A in a concentration c_{A(n)} and the parasitic species P in a concentration c_{P(n)f}, the value c_{P(n)f} having a non-zero deviation Δc_{P(n)} from the value c_{P(n)i};
determining a measurement signal S_{(n,m)}(t) during the fluid injection step representative of interactions of the analyte A and of the parasitic species P with receptors, for each of the sensitive sites M;
determining first signatures Su_{(n,m)f} from the measurement signal S_{(n,m)}(tϵPh2) associated with the gas sample, which has been corrected by a reference value S_{(n,m)i} from the measurement signal S_{(n,m)}(tϵPh1) associated with the carrier gas;
determining a difference Δc_{P(n)} in concentration of the parasitic species P between the first phase Ph1 and the second phase Ph2;
reiterating the preceding steps, by incrementing the rank n until N first signatures representative of interactions of the analyte A and the parasitic species P with the receptors are obtained, with N>1, the gas samples being such that the differences Δc_{P(n)} are different in pairs;
forming a matrix of first signatures Su_{A,P}, of dimensions N×M, formed from N first signatures Su_{(n,m)f} determined for the M sensitive sites; and of a relative concentration vector Δc_{P}, of dimension N×1, from determined N differences Δc_{P(n)};
determining an estimated solution {\hat{k}_{P|A}; ĉ_{A}; \hat{k}_{A}}; the product of which ĉ_{A}\hat{k}_{A}^{T} forms a matrix of corrected signatures Suc_{A} characterising the analyte A present in the N gas samples, the estimated solution:
minimising the cost function f=Su_{A,P}−Δc_{P}k_{P|A}−c_{A}k_{A}^{T}, and
maximising the cost function g=Su_{A,P}−Δc_{P}k_{P|A}^{T}: where
c_{A}, k_{A} and k_{P|A} are variables defined as follows:
c_{A} is a concentration vector of analyte A, of dimension N, formed from N concentration values c_{A(n)} of the gas samples,
k_{A} is an affinity vector of the analyte A, of dimension M, formed from the M values of an interaction affinity of the analyte A with the receptors of the sensitive sites,
K_{P|A} is is an affinity vector of the parasitic chemical species P, of dimension M, formed from the M values of an interaction affinity of the parasitic chemical species P with the receptors of the sensitive sites in the presence of the analyte A.

2. The charterisation method according to claim 1, wherein the step of determining the estimated solution carries out the minimisation of the objective function f and the maximisation of the objective function g at the same time, the values of the relative concentration vector Δc_{P} all being positive.

3. The charterisation method according to claim 2, wherein the step of determining the estimated solution is performed by an iterative algorithm, of iteration indicator i.

4. The charterisation method according to claim 3, wherein the step of determining the estimated solution includes a substep of determining the value \hat{k}_{P|A}⁽ⁱ⁺¹⁾ of the variable k_{P|A}, given the value ĉ_{A}⁽ⁱ⁾ of the variable c_{A} and of the value \hat{k}_{A}⁽ⁱ⁾ of the variable k_{A}, by a fixed point method.

5. The charterisation method according to claim 4, wherein the step of determining the estimated solution includes a substep of determining the value ĉ_{A}⁽ⁱ⁺¹⁾ of the variable c_{A} and of the value \hat{k}_{A}⁽ⁱ⁺¹⁾ of the variable k_{A}, given the value \hat{k}_{P|A}⁽ⁱ⁺¹⁾ of the variable kps having been determined by a singular value decomposition of a matrix R=Su_{A,P}−Δc_{P}k_{P|A}^{T(i+1)}.

6. The charterisation method according to claim 1, wherein the step of determining the estimated solution firstly includes a substep of minimising the objective function f to obtain a plurality of Q local solutions minimising the objective function f, with Q>1, followed by a substep of maximising the objective function g.

7. The charterisation method according to claim 6, wherein the minimising substep provides Q local solutions each formed by estimates \hat{k}_{P|A}^{T(q)}, ĉ_{A}^{(q)}, \hat{k}_{A}^{T(q)}, of rank q ranging from 1 to Q, of variables k_{P|A}, c_{A}, and k_{A}.

8. The charterisation method according to claim 7, wherein the maximising substep includes determining corrected signature Q matrices Suc_{A}^{(q)} such that: ∀qϵ[1, Q], Suc_{A}^{(q)}=Su_{A,P}−Δc_{P}\hat{k}_{P|A}^{T(q)}.

9. The charterisation method according to claim 8, including, following the determination of the corrected signature Q matrices Suc_{A}^{(q)}, normalising each of the corrected signature matricesd Suc_{A}^{(q)} to obtain normalised corrected signature Q matrices Sucn_{A}^{(q)}.

10. The charterisation method according to claim 9, including, following the determination of the corrected and normalised signature Q matrices Sucn_{A}^{(q)}, determining variance score, for each of the Q matrices Sucn_{A}^{(q)}, the variance score being defined as the trace of the covariance matrix for each of the Q matrices Sucn_{A}^{(q)}, the matrix Sucn_{A}^{(qf)} having a minimal score characterising the analyte A present in the N gas samples.

11. The charterisation method according to claim 8, including, following the determination of the corrected signature Q matrices Suc_{A}^{(q)}, determining a norm of each of the Q matrices Suc_{A}^{(q)}, followed by an identification of the matrix Suc_{A}^{(qf)} having the maximum norm.

12. The charterisation method according to claim 11, wherein the matrix Suc_{A}^{(qf)} is normalised, thus providing a matrix Sucn_{A}^{(qf)} on characterising the analyte A present in the N gas samples.

13. The charterisation method according to claim 1, wherein the electronic nose includes a device for measuring interactions by adsorption/desorption of the surface plasmon resonance optical type or of the Mach-Zehnder interferometry type.

14. The charterisation method according to claim 1, wherein the electronic nose includes a device for measuring interactions by adsorption/desorption of the resistive, piezoelectric, mechanical or acoustic type.

15. The charterisation method according to claim 1, wherein the electronic nose includes a fluid supply device configured to perform the fluid injection step, a measurement device configured to perform the step of determining the measurement signal, a sensor for measuring and determining the relative concentration of the parasitic chemical species, and a processing unit configured for implementing the step of determining the estimated solution.
